(19) United States
(12) Patent Application Publication
(10) Pub. No.: US 2016/0128699 A1
(43) Pub. Date: May 12, 2016

(54) HEX CAPTURE FOR SELF EXPANDING AND DRIVEN OCCLUDER

(71) Applicant: Cook Medical Technologies LLC, Bloomington, IN (US)

(72) Inventors: Richard Hadley, Otterbein, IN (US); Kevin Wilger, Lafayette, IN (US); Ryan Bradway, Tacoma, WA (US)

(21) Appl. No.: 14/884,905

(22) Filed: Oct. 16, 2015

Related U.S. Application Data
(60) Provisional application No. 62/078,710, filed on Nov. 12, 2014.

(51) Int. Cl. A61B 17/12

(52) U.S. Cl. CPC A61B 17/12172; A61B 17/12109; A61F 2/01

(57) ABSTRACT

A device for deploying, adjusting, and retrieving a medical implant is described. The device has at least one wing with a receiving element formed thereon for engaging the medical implant. An outer sheath may be used to move the device from the open configuration to the closed configuration. A method for using such a device is also provided.

### RELATED APPLICATIONS

This application claims the benefit of priority under 35 U.S.C. §119(a) to U.S. Provisional Application No. 62/078,710, filed on Nov. 12, 2014, which is incorporated by referenced here in its entirety.

### BACKGROUND OF THE INVENTION

1. Field of Invention

The present invention relates generally to medical devices. More specifically, the invention relates to an apparatus for deploying, adjusting, and retrieving a medical implant to or from a body cavity, and also provides a method for retrieval of a medical implant.

2. Background

In cases where it is necessary to occlude a vessel or other body cavity, or place a different medical implant, there can be a need for a device that would allow a physician to resheath and reposition the medical implant before nesting is complete. Some areas of the anatomy are tortuous and as a result require a more controlled repositioning ability. By utilizing a delivery device with the ability to matingly engage a portion of or the entire implant, recapture and relocation of the device such that optimal placement and maximum efficacy of the implant are ensured are facilitated.

Similarly, retrieval of a medical implant, such as a filter or an occlusion device, from a body cavity is a high-precision process which can be difficult to achieve in tortuous portions of the anatomy. Care must be taken when introducing a retrieval device to the body of a patient, and often after the device engages the implant, the means of attachment of the implant to the anatomy of the patient must be disrupted. Sometimes the most effective way of doing so is by providing a rotational motion to loosen bonds between the implant and the anatomy.

There is a need for improved delivery and retrieval devices and methods for manipulating medical implants that benefit from controlled deployment, repositioning, or rotational motion.

### BRIEF SUMMARY OF THE INVENTION

In one embodiment, a device for delivery and retrieval of a medical implant is described. The device comprises a pusher member including a receiving portion. The pusher member comprises a stem. The stem comprises a first end and extends to a second end, the stem being tubular and through which a longitudinal axis is defined. The pusher member also comprises at least one wing having a proximal end attached to the second end of the stem and extending distally to a distal end, the at least one wing having a recessed portion adjacent the distal end. The pusher member has an open configuration to allow a medical implant to be disposed longitudinally between the recessed portion of the at least one wing and a receiving portion of the pusher member. The pusher member has a closed configuration so that the recessed portion and the receiving portion cooperate to matingly receive the medical implant.

In another embodiment, a system for delivery and retrieval of a medical implant is provided. The system comprises a medical implant comprising a first end extending to a second end, and a pusher member. The pusher member comprises a stem comprising a third end and extending to a fourth end, the stem being tubular and through which a longitudinal axis is defined. Further, there is at least one wing having a proximal end attached to the fourth end of the stem and extending distally to a distal end, the at least one wing having a recessed portion adjacent the distal end. The pusher member has an open configuration to allow a medical implant to be disposed longitudinally between the recessed portion of the at least one wing and a receiving portion of the pusher member. The pusher member additionally has a closed configuration so that the recessed portion and the receiving portion form a female shape and cooperate to matingly receive the first end of the medical implant.

In a further embodiment, a method of retrieving a medical implant having a first end and a second end from a body cavity of a patient. In a first step, the method comprises providing a device comprising a pusher member. The pusher member comprises a stem comprising a third end and extending to a fourth end, the stem being tubular and through which a longitudinal axis is defined. The pusher member comprises a plurality of wings, each wing having a proximal end attached to the fourth end of the stem and extending distally to a distal end. Each wing has a recessed portion adjacent the distal end. The plurality of wings has an open configuration to allow a medical implant to be disposed longitudinally between the recessed portions. The plurality of wings has a closed configuration so that the recessed portions cooperate to matingly receive the medical implant. The device further comprises a tubular member attached to the third end of the stem, the tubular member having a second lumen formed therein and in fluid communication with the first lumen.

In a second step, the method comprises providing an outer sheath comprising a fifth end and extending to a sixth end, the outer sheath being tubular having a second inner wall defining a third lumen formed therein, wherein the pusher member is held within the third lumen in the closed configuration.

In a third step, the method comprises introducing the outer sheath into a body cavity of a patient.

In a fourth step, the method comprises advancing the outer sheath distally to a position proximal of the medical implant.

In a fifth step, the method comprises advancing the plurality of wings distally from the sixth end of the outer sheath to the open configuration, the recessed portions being disposed about the first end of the medical implant.

In a sixth step, the method comprises advancing the outer sheath distally to slidably dispose the plurality of wings within the third lumen in the closed configuration, the recessed portions cooperating to matingly receive the first end of the medical implant.

In a final step, the method comprises retracting the outer sheath proximally to remove the device and the medical implant from the patient.

Further objects, features, and advantages of the present invention will become apparent from consideration of the following description and the appended claims when taken in connection with the accompanying drawings.

### BRIEF DESCRIPTION OF THE DRAWINGS

FIG. 1A is a perspective view of a pusher member in its open configuration in accordance with the principles of one embodiment of the present invention;

FIG. 1B is a perspective view of the pusher member of FIG. 1A in its closed configuration;

FIG. 1C is a close-up perspective view of one of the wings of the pusher member of FIGS. 1A-1B;

FIG. 2A is a perspective view of a device including a pusher member in accordance with another embodiment of the present invention;

FIG. 2B is a side view of the pusher member of FIG. 2A in its open configuration;

FIGS. 2C-2E are views of the ends and receiving portions of devices in accordance with further embodiments of the present invention;

FIGS. 3A-3D are perspective views of devices with more than two wings in accordance with further embodiments of the present invention;

FIG. 4A is a perspective view of a medical implant for use with a system according to one embodiment of the present invention;

FIG. 4B is a perspective view of a system according to one embodiment of the present invention in which the medical implant of FIG. 4A is engaged with the pusher member in its closed configuration;

FIG. 4C is a side view of the system of FIG. 4B wherein the pusher member is in its open configuration and the proximal end of the medical implant is disposed between the wings of the pusher member;

FIG. 5 is a schematic view of a tubular member and a pusher member formed as separate parts in accordance with a further embodiment of the present invention; and

FIG. 6 is a flow chart of a method for using a device having a pusher member to retrieve a medical implant in accordance with another embodiment of the present invention.

### DETAILED DESCRIPTION OF THE INVENTION

The description that follows is not intended to limit the scope of the invention in any manner, but rather serves to enable those skilled in the art to make and use the invention.

It is to be understood that the figures are schematic and do not show the various components to their actual scale. In many instances, the figures show scaled up components to assist the reader.

In this description, when referring to a device, a catheter, or a medical implant, the term distal is used to refer to an end of a component which in use is furthest from the physician during the medical procedure, including within a patient. The term proximal is used to refer to an end of a component closest to the physician and in practice in or adjacent an external manipulation part of the deployment or treatment apparatus.

The terms “substantially” or “about” used herein with reference to a quantity includes variations in the recited quantity that are equivalent to the quantity recited, such as an amount that is equivalent to the quantity recited for an intended purpose or function. In the case of a numerical quantity, the terms “substantially” or “about” shall mean a range consisting of a value 50% less than the recited value to a value 50% greater than the recited value, inclusive.

The term “adjacent” as used herein with reference to the position of an object or a portion thereof refers to the proximity of two structures or elements. Particularly, elements that are identified as being “adjacent” may be either abutting or fluidly connected. Such elements may also be near or close to each other without necessarily contacting each other. The term “adjacent” or “adjacent to” can include meanings not limited to “next to,” “adjoining,” “in contact with,” and “in proximity to.” In some instances, adjacent components are separated from one another by one or more intervening portions of an object.

The term “disposed between” as used herein, when used with reference to the position of an object, means that an object or a portion thereof occupies a space between that which it is disposed between. This encompasses situations wherein the object is in physical contact with that which it is disposed between as well as situations wherein it does not contact that which it is disposed between.

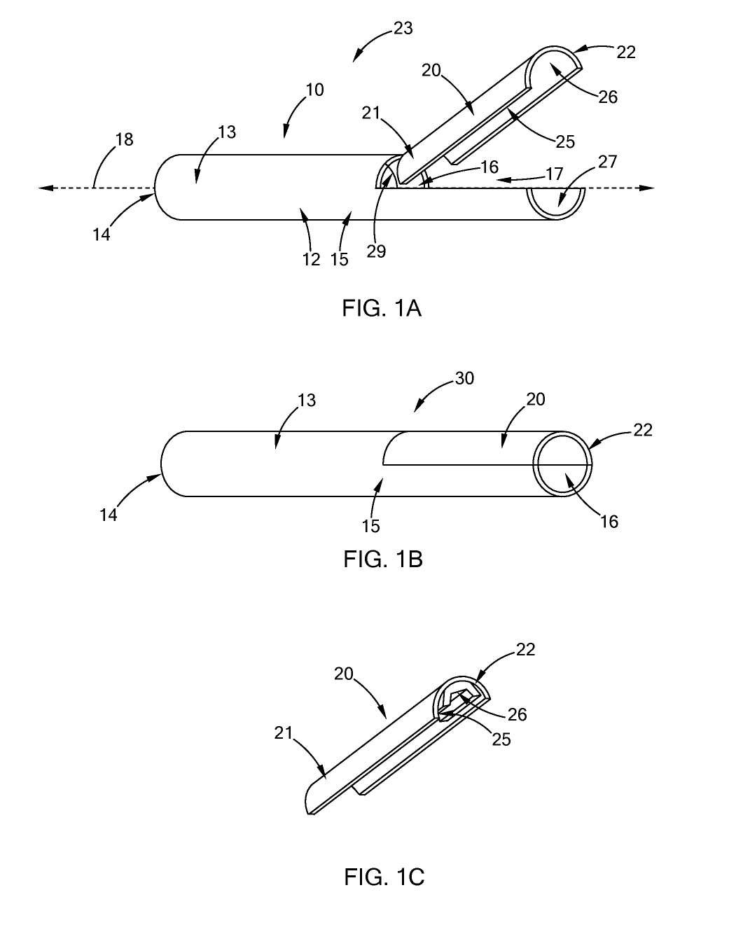

FIG. 1A shows a device in accordance with one embodiment of the present invention. The device 10 comprises a pusher member 12. The pusher member 12 comprises a stem 13, which extends from a first end 14 to a second end 15, and a wing 20. The stem is tubular in shape and has an outer wall and an inner wall 16. In the illustrated embodiment, the stem 13 has a first lumen 17 formed therein. The first lumen 17 can extend through the entirety of the stem 13, or through a portion of it. A longitudinal axis 18 is formed through first lumen 17.

In another embodiment, the stem may comprise a solid tubular or cylindrical piece. Optionally, the stem may have a groove formed thereon for providing a surface on which to engage a wire guide.

The wing 20 has a proximal end 21 and extends to a distal end 22. The proximal end 21 is attached to the second end 15 of the stem. The wing is movable and is hingedly connected to the stem 13. The wing 20 can have a curved structure, the wing 20 being concave with the cavity surrounding the longitudinal axis 18. In one embodiment, the cross section of the wing has a semicircular shape. The inner cavity of the wing is recessed portion 25.

The pusher member 12 has an open configuration 23, as illustrated in FIG. 1A. In such a configuration, the wing 20 is positioned such that distal end 22 is moved away from the remainder of the stem. This creates a space between the wing 20 and the stem 12 where an end of a medical implant can be positioned. When the medical implant is properly positioned, the pusher member 12 can be moved into its closed configuration 30, as seen in FIG. 1B.

As seen in FIG. 1C, a receiving element 26 is present on the pusher member 12. In the illustrated embodiment, the receiving element 26 is on recessed portion 25 of the wing. In one embodiment, the receiving element 26 may simply be all of or part of the recessed portion of the wing. A receiving portion 27 may also be a portion of the stem in the one-winged embodiment of FIG. 1A. Such a receiving portion 27 is where the end of the medical device comes to rest when in the closed configuration.

In another embodiment, the receiving portion 26 may constitute an additional element present on the pusher member 12. In the case of the illustrated embodiment of FIG. 1C, the receiving portion is an added component on recessed portion 25 and consists of a tripartite structure. One segment of this tripartite structure lies against the recessed portion 25 of the wing 20 and is centered between two segments of similar length, each forming an angle of approximately 120 degrees with the central segment and pointing into the first lumen 17. The receiving portion 26 thus has a half-hexagon shape in the illustrated embodiment of FIG. 1C.

In one embodiment, the receiving portion 26 is formed unitarily or monolithically with the wing 20. In another embodiment, the receiving portion 26 is formed as a separate part and is attached to the wing 20 by any conventional means, such as by adhesive or by soldering.

It is to be understood that the wing or the wings, being a specific portion of the pusher member, may have a receiving element thereon which is equivalent to a receiving portion of the stem. In the case of a one-wing device wherein the medical implant is positioned between the wing and a static or minimally-moving portion of the pusher member, the receiving portion will be on the unhinged, relatively static portion of the pusher member. In the case of a multi-wing device wherein the medical implant is surrounded by two or more wings, it is possible that the receiving portion is a receiving element have a receiving element thereon which is equivalent to a receiving portion of the stem. In the case of a one-wing device wherein the medical implant is positioned between the wing and a static or minimally-moving portion of the pusher member, the receiving portion will be on the unhinged, relatively static portion of the pusher member. In the case of a multi-wing device wherein the medical implant is surrounded by two or more wings, it is possible that the receiving portion is a receiving element of any of the plurality of wings.

In another embodiment, a small spring may be provided at the hinge region, which will cause the wing to open away from the body of the device when unconstrained.

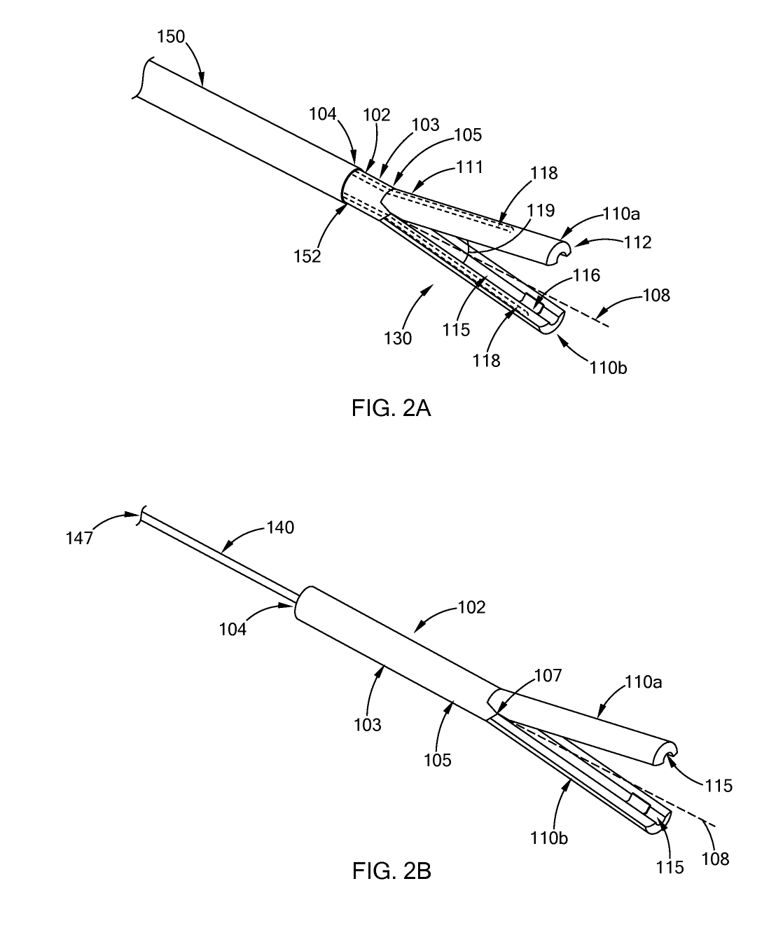

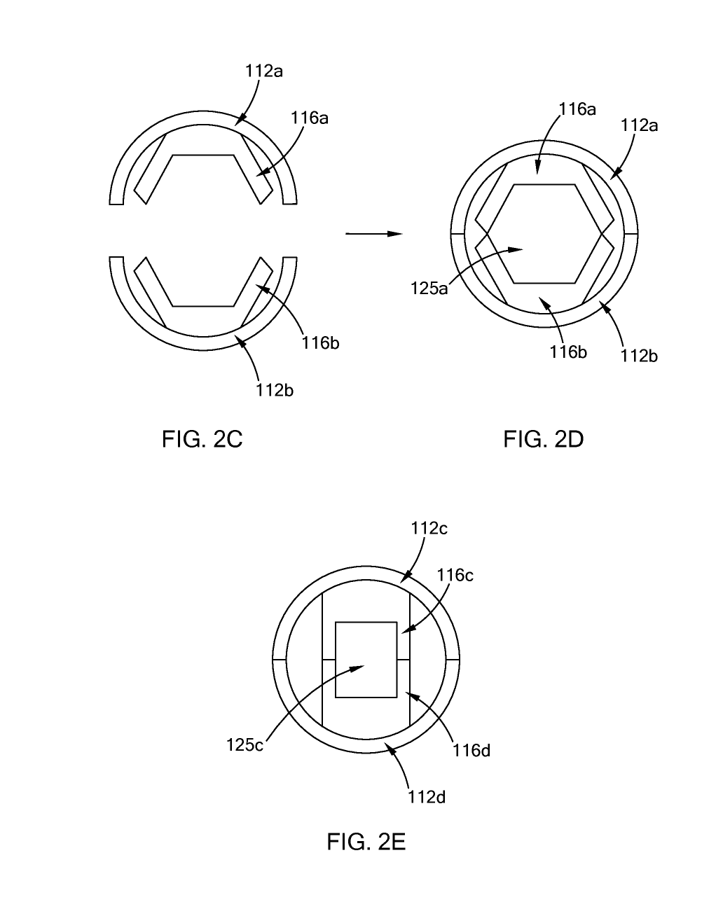

Another device in accordance with the principles of the present invention is depicted in FIG. 2A. The device 101 comprises a pusher member 102 having stem 103. The stem 103 extends from first end 104 and second end 105. The stem 103 is tubular in construction and has an outer wall, an inner wall 106, and a first lumen 107 through which a longitudinal axis 108 is defined.

In the embodiment of FIG. 2A, the pusher member 102 further comprises a plurality of wings 110a/110b. In the illustrated embodiment, the plurality of wings consists of two wings. The wings 110a/110b each have a proximal end 111 which is attached to the second end 105 of the stem 103, and extend to a distal end 112. The wings have an open configuration 130 in which the wings 110a and 110b are set at an angle to the longitudinal axis 108. In one embodiment, this angle between a wing and the longitudinal axis may be about 15 degrees. Therefore, the angle 119 between wing 110a and wing 110b is, in this embodiment, about 30 degrees.

The device 101 as depicted in the embodiment of FIG. 2A further comprises an outer sheath 150. The outer sheath 150 is of a tubular construction and has a third lumen 157 formed therethrough. The pusher member 102 is movable within the outer sheath 150, and is rotatable and slidable. When the pusher member 102 is drawn into the third lumen of the outer sheath 150, the wings 110a and 110b come together and the pusher member adopts its closed configuration 120.

In one embodiment, the device 101 may further comprise reinforcing elements 118. The reinforcement elements 118 are rod-like structures that are designed to stabilize the wings and bias them into their open configuration when unconstrained. In a first embodiment, each reinforcing element 118 starts from within the wall of the stem 103 (that is, embedded within the solid portion between the outer wall and the inner wall 106) and extends into each of the plurality of wings 110a/110b. Alternative embodiments of a device in accordance with the principles of the present invention include structures wherein the reinforcing elements 118 run along the outer walls of the stem or the wings, or along the inner walls of the stem or the wings, or a combination of these configurations.

The reinforcing elements may comprise at least one shape memory material. Shape memory materials reversibly transform between a lower temperature phase (martensite) and a higher temperature phase (austenite) while passing through a transition temperature therebetween. Shape memory alloys have the desirable property of becoming rigid when heated above the transition temperature.

A shape memory alloy suitable for the present invention is an alloy comprising nickel and titanium. When a nickel-titanium shape memory alloy is heated above the transition temperature, the material undergoes a phase transformation from martensite to austenite, such that the material starts with a substantial amount of elasticity but at the transition temperature begins to become rigid. The transition temperature is dependent on the relative proportions of the alloying elements nickel (Ni) and titanium (Ti) and the optional inclusion of alloying additives. Often the proportions of Ni and Ti are selected so that the material is austenite at body temperature.

The reinforcing elements 118, therefore, would be biased into a straight segment designed to move the device to its open configuration 130 when the device is unconstrained, particularly when the device is not being held within the third lumen 157 of outer sheath 150. The reinforcing elements would pass from the stem 103, through the hinged portion formed between the second end 105 of the stem and the proximal end 111 of the wing 110, and into the solid portion of the wing 110 to achieve this effect.

The device as illustrated in the embodiment of FIG. 2B further comprises a tubular member 140 which lies proximal to first end 104 of the pusher member. The tubular member 140 has a second lumen 147 formed therein or therethrough. The stem 103 of pusher member 102 has a first diameter, and the tubular member 140 has a second diameter smaller than the first diameter. Second lumen 147 is in fluid connection with first lumen 107 so that fluids or solids can be passed between tubular member 140 and pusher member 102.

The tubular member 140 is a resilient member which is still flexible enough to navigate tortuous anatomy. Its smaller second diameter allows it to be more readily manipulated by the physician using the device and is, in one embodiment, long enough to extend all the way to the end closest to the physician. In one embodiment, the tubular member 140 is made of stainless steel and is a hollow cannula with a diameter of about 1.5 millimeters (mm).

The outer sheath 150 has a third diameter which is larger than the overall profile of the pusher member 102 and the tubular member 140. The outer sheath 150 should be made of a material such that pusher member 102 and tubular member 140 are able to be rotated within the third lumen of the device, and so that the distal ends of the wings 110 are able to be slid out of the third lumen 157 of the outer sheath 150.

FIG. 2C depicts an end view of wings 110a/110b. At the distal ends 112a/112b of wings 110a/110b are receiving elements 116a/116b. As in the embodiment of FIG. 1, the receiving elements 116a/116b are attached to or formed as a portion of the recessed portions of the wings 110a/110b and have three-segment structures forming half-hexagons, with about 120 degree angles between connected segments. FIG. 2C depicts the pusher member in its open position 130. If the distal ends 112a/112b are brought together, the pusher member 102 adopts its closed position 120, as shown in FIG. 2D. As a result, the receiving elements 116a/116b come together to form a fully hexagonal shape which can function as a female-shaped receiving element. While a number of different medical implant designs would allow for capture in this hexagonal female shape, it is particularly well-suited to capture a hexagonal male portion. A female shape has at least some space into which a corresponding male portion can be inserted.

Other shapes for female captures are envisioned. In FIG. 2E, distal ends 112c/112d come together in the closed configuration to bring receiving elements 116c/116d into close proximity with one another. The 90 degree bends in receiving elements 116c/116d cause the female capture to take on a square shape. Many other shapes for the female capture are possible, including triangular, rectangular, pentagonal, octagonal, and any convex or concave shape suitable for receiving a compatible portion of a medical implant.

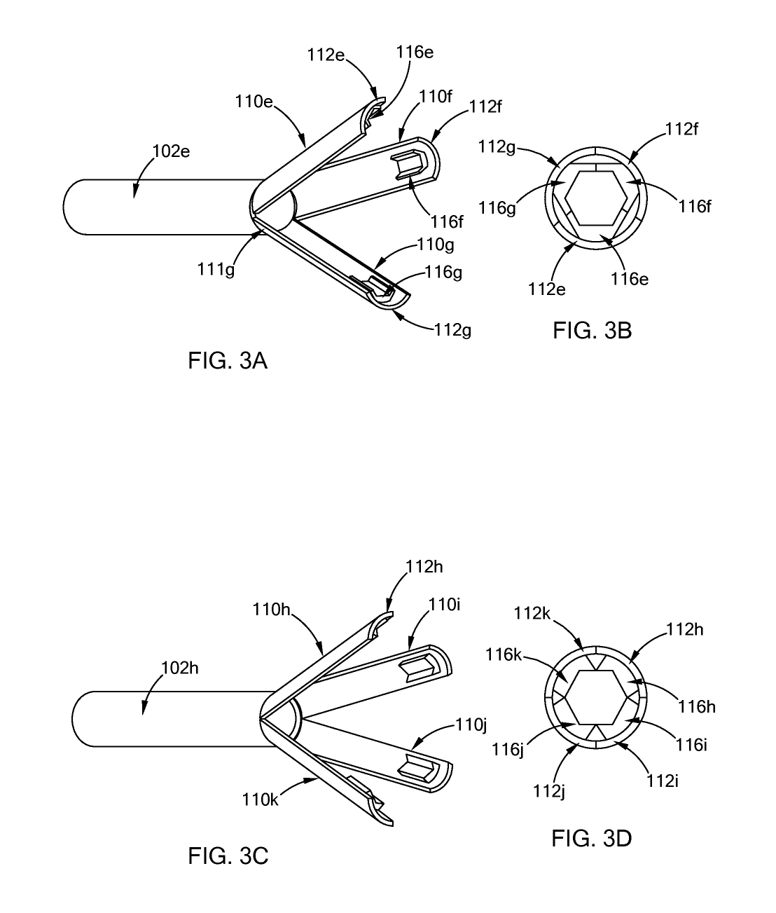

Turning to FIG. 3A-3D, further embodiments of devices in accordance with the principles of the present invention are shown. Specifically, FIG. 3A and 3B depict a device having three wings and FIGS. 3C and 3D depict a device having four wings. These devices function largely in the same manner as one- or two-wing devices. The three wing device of FIG. 3A has wings 110e/110f/110g positioned at 120 degree angles around the circumference of the pusher member. The end view depicted in FIG. 3B shows one way that a hexagonal female receiving shape could be constructed; in this embodiment, the outer segments of receiving elements 116e/116f/116g are substantially shorter than the central segments, and this is what permits them to come together in the closed position to create a fully hexagonal shape for receiving medical implants.

The four-wing device of FIG. 3C and FIG. 3D has wings 110h/110i/110j/110k spread around the circumference of the pusher member at intervals of about 90 degrees between adjacent wings. The female portion would be made up of four receiving elements in this case. As illustrated, four receiving elements 116h/116i/116j/116k form a typical hexagon, this time doing so by offering only two fragments for quadrant (wing) and retaining the 120 degree angle therebetween. However, the receiving female shape can take any suitable shape.

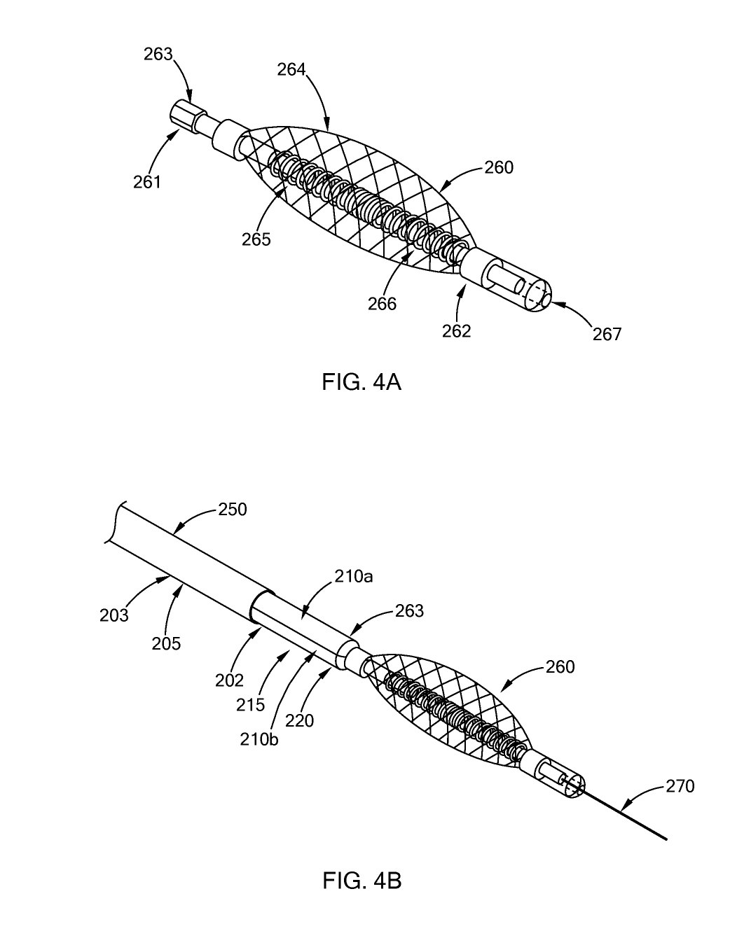

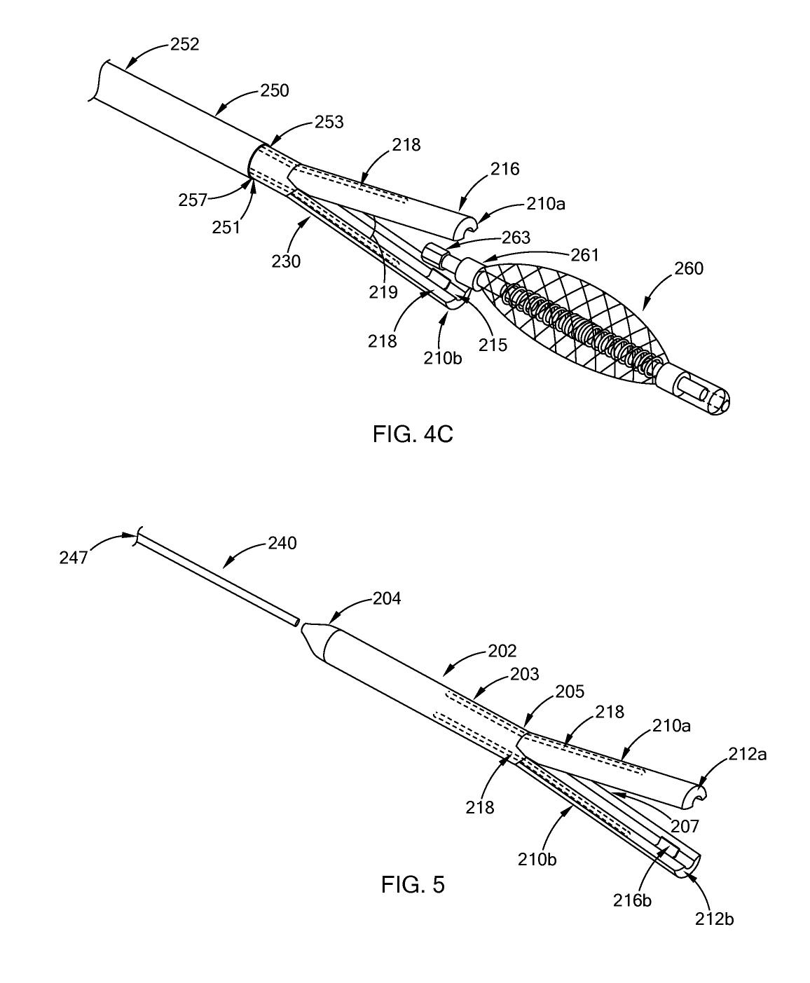

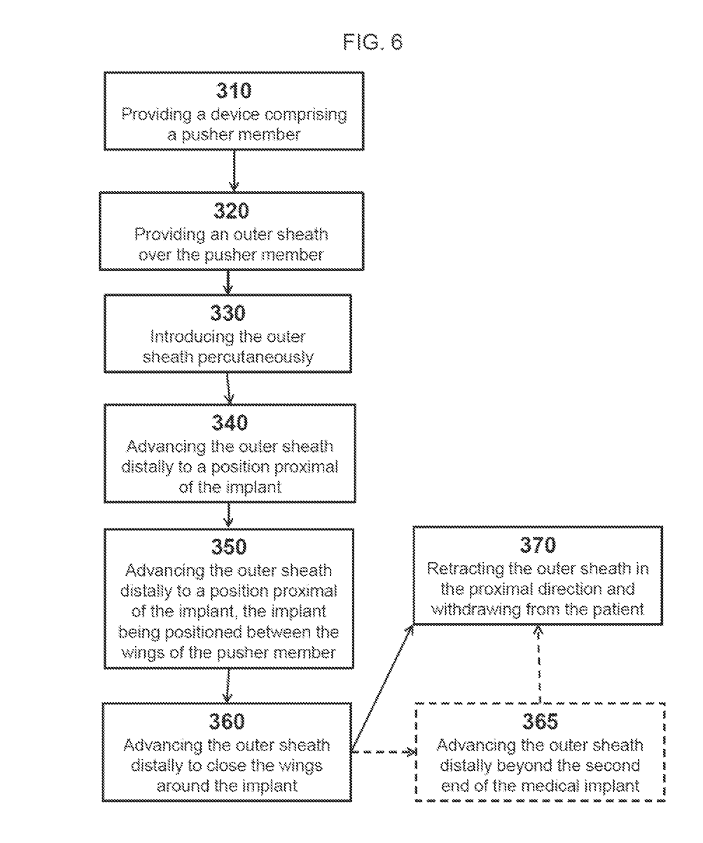

Turning to FIG. 4A, an exemplary medical implant 260 for use in a system with a device according to the present disclosure is illustrated. The implant 260 has an inner coil spring which passes through the implant. This spring is configured to contract in the absence of an outside pressure or stretching force. It extends through mesh lumen from proximal collet at first end 261 and extends to a distal collet at second end 262.

The implant 260 of FIG. 4A is expanded by a screw mechanism. The male shape 263, in this case a hex nut, at first end 261 can be contact by and held within a complementary female shape of the pusher member. The implant 260 allows the practitioner to control expansion of the device by rotating a member of the delivery system. The device can have a fourth lumen 267 formed therein or therethrough, allowing deployment of the implant over a wire guide 270.

FIG. 4B shows the implant 260 of FIG. 4A engaged with the device 201 with the pusher member 202 in its closed configuration 220. In this illustration, the outer sheath 250, which extends from fifth end 251 to sixth end 252 and has a tubular shape and a second inner wall 253, with a third lumen 257 formed therein, has been extended past the fourth end 205 of the pusher member 202 and as such as forced the wings 210 closed. This in turn causes the pusher member 202 to move to its closed configuration 220 and thus traps the medical implant 260 therein. This device could then be deployed, retrieved, or adjusted if it were to be put in this position during a treatment procedure.

FIG. 4C shows the same device 201 but in the open configuration 230. The outer sheath 250 in this case has been withdrawn proximally and the wings are no longer in any part within third lumen 257. The wings 210 are biased into the open configuration 230 and moving to this configuration ensures release of the medical implant 260.

In an embodiment of this invention, the medical implant of FIG. 4 could have a fourth lumen 267 formed therethrough. Such a lumen would ensure that an opening coincident with and surrounding the longitudinal axis 208 of the system would be vacant space of a size and shape that a wire guide 270 could be fed therethrough. The wire guide 270 would be advanced through the body cavity of the patient until the site of deployment for the medical implant 260 is reached. Then the implant 260 could be deployed over the wire.

A further embodiment of a device in accordance with the present invention is shown in FIG. 5. In this case, the device 201 comprises pusher member 202 as its own piece separate from the tubular member 240. The pusher member 202 could be formed separately and then attached in fluid communication with any suitable, resilient, flexible tube and use that to retrieve.

In another embodiment, the present invention is a method of retrieving retrieving a medical implant having a first end and a second end from a body cavity of a patient. The method comprises a first step 310 of providing a device comprising a pusher member. The pusher member in turn comprises a stem which has a third end and extends to a fourth end. The stem is tubular and has a first inner wall defining a first lumen formed therein and through which a longitudinal axis is defined.

The pusher member also comprises a plurality of wings, each wing having a proximal end attached to the fourth end of the stem and extending distally to a distal end, each wing having a receiving element adjacent the distal end.

The plurality of wings has an open configuration to allow a medical implant to be disposed longitudinally between the receiving elements and a closed configuration so that the receiving elements cooperate to matingly receive the medical implant.

The device provided in step 310 has a tubular member attached to the third end of the stem. The tubular member has a second lumen formed therein and is in fluid communication with the first lumen.

In a second step 320, the method provides an outer sheath comprising a fifth end and extending to a sixth end. The outer sheath is tubular and has a second inner wall defining a third lumen formed therein, wherein the pusher member is held within the third lumen in the closed configuration.

In a third step 330, the method comprises introducing the outer sheath into a body cavity of a patient percutaneously. This step can be achieved by any conventional method, such as a variation on the Seldinger method, and can be done using any additional equipment, such as a dilator.

In a fourth step 340, the retrieval method comprises advancing the outer sheath distally to a position proximal of the medical implant. This positions the retrieval apparatus in such a way as to best affect retrieval of the implant.

In a fifth step 350, the method includes advancing the plurality of wings distally from the sixth end of the outer sheath to the open configuration, the receiving elements being disposed about the first end of the medical implant. The end of the implant is now spatially placed between the plurality of wings, or in the case of a single-wing device, between the static portion of the pusher member and the wing.

In a sixth step 360, the outer sheath is advanced distally to slidably dispose the plurality of wings within the third lumen in the closed configuration, the receiving elements cooperating to matingly receive the first end of the medical implant. In this step, as the outer sheath distally slides over the wings, the wings begin to adopt the closed configuration, and the angle between them decreases as the outer sheath moves further in the distal direction.

In a seventh step 370, the method concludes by retracting the outer sheath proximally to remove the device and the medical implant from the patient. The medical implant, having been securely gripped by the pusher member which is now in the closed position and residing within the third lumen of the outer sheath, is withdrawn from the patient. Different structures of a variety of implants can be used but changes to the method may be necessary to ensure that the implants can safely be extracted.

In an optional step, the method of retrieval may contain the step of advancing the outer sheath distally to slidably dispose the sixth end of the outer sheath to a position distal of the second end of the medical implant. For some implants, it may be necessary to not only use the outer sheath to return the pusher member to its closed configuration, but also to receive the medical device into the lumen of the outer sheath itself. This is particularly true of implants that are of flexible construction and which have a larger diameter than the outer sheath. The implant is compressed and easily removed from the body when it is in the lumen of the outer sheath. In another related embodiment, the sixth end is not slidably disposed distal to the second end of the implant, but rather encompasses just a portion of the medical implant.

Additionally, the pusher member and the tubular member should be movable to some extent within the third lumen of the outer sheath. This is particularly important for devices which expand or contract due to rotational motion. The device may need to be first gripped by the pusher member and then rotated to bring it down to size in the retrieval process. In another embodiment, during a related deployment method, the medical implant may not fully expand unless subjected to rotational motion while at least partway within the third lumen of the outer sheath. Therefore, movability within the outer sheath, including rotational and sliding motion, can be desirable.

This device and method allow for greater control of the screw-driven adjusting feature of various occlusion devices and controlled delivery within tortuous areas when nesting. Further, they impart compatibility with over-wire delivery of devices.

Such a device and a method can be advantageous due to the shorter procedure times they impart on deployment or retrieval of a medical implant. Because the advancing or retraction of an outer sheath is all that is required to affect complete deployment of a device, the effect of implantation (for instance, occlusion of a blood vessel) can be realized almost immediately. In the case of retraction, the positive interaction between the male shape at the end of the implant and the complementary female shape which constitutes a receiving space in the retrieval device allows for a sure connection and improved confidence in retrieval.

While the apparatus of the invention has been described above with reference to certain specific embodiments thereof, it is to be clearly understood that these embodiments have been given for purposes of illustration only and are not intended to be limiting. The scope of the invention is bounded only by the scope of the claims which are set out hereafter.

1. A device for delivery and retrieval of a medical implant, the device comprising:
a pusher member including a receiving portion, the pusher member comprising:
a stem comprising a first end and extending to a second end, the stem being tubular and defining a longitudinal axis therethrough; and
at least one wing having a proximal end attached to the second end of the stem and extending distally to a distal end, the at least one wing having a recessed portion, the at least one wing having a receiving element on the recessed portion and adjacent the distal end;
the pusher member having an open configuration to allow a medical implant to be disposed longitudinally between the recessed portion of the at least one wing and a receiving portion of the pusher member;
the pusher member having a closed configuration so that the receiving element and the receiving portion cooperate to matingly receive the medical implant.

2. The device of claim 1 further comprising a plurality of wings, each of the plurality of wings having a proximal end attached to the second end of the stem and extending distally to a distal end, a least one of the plurality of wings having a receiving element adjacent the distal end, wherein the receiving portion of the pusher member comprises the receiving element of the at least one of the plurality of wings.

3. The device of claim 1 wherein the stem has an inner wall defining a first lumen formed therein, the device further comprising a tubular member attached to the first end of the stem, the tubular member having a second lumen formed therein and in fluid communication with the first lumen.

4. The device of claim 1 further comprising a tubular outer sheath having a third lumen formed therein, the at least one wing being movably disposed within the third lumen in the closed configuration.

5. The device of claim 4 wherein the at least one wing extends distally from the outer tubular sheath in the open configuration.

6. The device of claim 1 wherein the receiving element and the receiving portion cooperate to form a female shape and matingly receive the medical implant.

7. The device of claim 1 further comprising plurality of reinforcing elements, each reinforcing element being disposed within the stem and the at least one wing, each reinforcing element extending distally from the stem and being solely disposed within one wing.

8. The device of claim 7 wherein each reinforcing element comprises a shape memory material such that the at least one wing is biased in the open configuration.

9. The device of claim 7 wherein the at least one wing extends distally from the stem at an angle of about 15 degrees relative to the longitudinal axis in the open configuration.

10. The device of claim 1 further comprising:
a plurality of wings, each of the plurality of wings having a proximal end attached to the second end of the stem and extending distally to a distal end, at least one of the plurality of wings having a receiving element adjacent the distal end, wherein the receiving portion of the pusher member comprises the receiving element of the at least one of the plurality of wings;
a tubular member attached to the first end of the stem, the tubular member having a second lumen formed therein and in fluid communication with the first lumen;
a tubular outer sheath having a third lumen formed therein, the at least one wing being movably disposed within the third lumen in the closed configuration;
a plurality of reinforcing elements, each reinforcing element being disposed within the stem and within each of the plurality of wings, each reinforcing element extending distally from the stem and being solely disposed within one wing, the reinforcing elements comprising a shape memory material;
wherein each of the plurality of wings extends distally from the outer tubular sheath in the open configuration, each of the plurality of wings extending distally from the stem at an angle of about 15 degrees relative to the longitudinal axis in the open configuration;
wherein the stem has an inner wall defining a first lumen formed therein; and
wherein the receiving element and the receiving portion cooperate to form a female shape and matingly receive the medical implant.

11. A system for delivery and retrieval of a medical implant, the system comprising:
a medical implant comprising a first end extending to a second end; and
a pusher member including a receiving portion, the pusher member comprising:
a stem comprising a third end and extending to a fourth end, the stem being tubular and defining a longitudinal axis therethrough; and at least one wing having a proximal end attached to the fourth end of the stem and extending distally to a distal end, the at least one wing having a receiving element adjacent the distal end;
the pusher member having an open configuration to allow a medical implant to be disposed longitudinally between the receiving element of the at least one wing and a receiving portion of the pusher member;
the pusher member having a closed configuration so that the receiving element and the receiving portion form a female shape and cooperate to matingly receive the first end of the medical implant.

12. The system of claim 11 further comprising a plurality of wings, each of the plurality of wings having a proximal end attached to the fourth end of the stem and extending distally to a distal end, at least one of the plurality of wings having a receiving element adjacent the distal end, wherein the receiving portion of the pusher member comprises the receiving element of the at least one of the plurality of wings.

13. The system of claim 11 wherein the first end of the medical implant comprises a male shape.

14. The system of claim 12 wherein the male shape is disposed within the female shape of the at least one receiving element and the receiving portion to matingly receive the medical implant in the closed configuration.

15. The system of claim 11 in which the stem has an inner wall defining a first lumen formed therein, the system further comprising a tubular member attached to the third end of the stem, the tubular member having a second lumen formed therein and in fluid communication with the first lumen.

16. The system of claim 11 further comprising a tubular outer sheath having a third lumen formed therein, the at least one wing being movably disposed within the third lumen in the closed configuration.

17. The system of claim 11 wherein the medical implant comprises a fourth lumen formed through the first end and the second end of the medical implant.

18. The system of claim 11 further comprising a plurality of reinforcing elements, each reinforcing element being disposed within the stem and the at least one wing, each reinforcing element extending distally from the stem and being solely disposed within one wing.

19. The system of claim 11 further comprising:
a plurality of wings, each of the plurality of wings having a proximal end attached to the fourth end of the stem and extending distally to a distal end, at least one of the plurality of wings having a receiving element adjacent the distal end, wherein the receiving portion of the pusher member comprises the receiving element of the at least one of the plurality of wings;
a stem having an inner wall defining a first lumen formed therein;
a tubular member attached to the third end of the stem, the tubular member having a second lumen formed therein and in fluid communication with the first lumen;
a tubular outer sheath having a third lumen formed therein, the at least one wing being movably disposed within the third lumen in the closed configuration;
a plurality of reinforcing elements, each reinforcing element being disposed within the stem and the at least one wing, each reinforcing element extending distally from the stem and being solely disposed within one wing;
wherein the first end of the medical implant comprises a male shape, the medical implant comprises a fourth lumen formed through the first end and the second end of the medical implant; and
wherein the male shape is disposed within the female shape of the at least one recessed portion and the receiving portion to matingly receive the medical implant in the closed configuration.

20. A method of retrieving a medical implant having a first end and a second end from a body cavity of a patient comprising the steps of:
providing a device comprising:
a pusher member comprising:
a stem comprising a third end and extending to a fourth end, the stem being tubular and through which a longitudinal axis is defined; and
a plurality of wings, each wing having a proximal end attached to the fourth end of the stem and extending distally to a distal end, each wing having a receiving element adjacent the distal end;
the plurality of wings having an open configuration to allow a medical implant to be disposed longitudinally between the receiving elements;
the plurality of wings having a closed configuration so that the receiving elements cooperate to matingly receive the medical implant;
a tubular member attached to the third end of the stem, the tubular member having a second lumen formed therein and in fluid communication with the first lumen;
providing an outer sheath comprising a fifth end and extending to a sixth end, the outer sheath being tubular having a second inner wall defining a third lumen formed therein, wherein the pusher member is held within the third lumen in the closed configuration;
introducing the outer sheath into a body cavity of a patient;
advancing the outer sheath distally to a position proximal of the medical implant;
advancing the plurality of wings distally from the sixth end of the outer sheath to the open configuration, the receiving elements being disposed about the first end of the medical implant;
advancing the outer sheath distally to slidably dispose the plurality of wings within the third lumen in the closed configuration, the receiving elements cooperating to matingly receive the first end of the medical implant;
retracting the outer sheath proximally to remove the device and the medical implant from the patient.
